(19) United States
(12) Patent Application Publication
(10) Pub. No.: US 2024/0087322 A1
(43) Pub. Date: Mar. 14, 2024

(54) Processing Images to Associate with Stages of a Surgical Procedure

(71) Applicants: University of Washington, Seattle, WA (US); Seattle Children's Hospital d/b/a Seattle Children's Research Institute, Seattle, WA (US)

(72) Inventors: Daniel King, Seattle, WA (US); Blake Hannaford, Seattle, WA (US); Yangming Li, Seattle, WA (US); Lingga Adidharma, Seattle, WA (US); Randall Bly, Seattle, WA (US); Al-Waleed M. Abuzeid, Seattle, WA (US); Kristen S. Moe, Seattle, WA (US)

(21) Appl. No.: 18/463,683

(22) Filed: Sep. 8, 2023

Related U.S. Application Data
(60) Provisional application No. 63/375,174, filed on Sep. 9, 2022.

(51) Int. Cl. G06V 20/50; G06N 3/0464; G06N 3/047; G06N 3/088; G06T 7/70; G06V 10/764; G06V 10/774; G06V 10/82; G16H 70/20

(52) U.S. Cl. CPC G06V 20/50; G06N 3/0464; G06N 3/047; G06N 3/088; G06T 7/70; G06V 10/764; G06V 10/774; G06V 10/82; G16H 70/20; G06T 2207/10016; G06T 2207/20081; G06T 2207/20084; G06T 2207/30004; G06V 2201/034

(57) ABSTRACT

A method includes accessing training images that collectively depict multiple stages of a surgical procedure. The method also includes accessing labels that indicate, for each of the training images, characteristics of one or more surgical tools depicted and a stage of the multiple stages of the surgical procedure depicted. The method also includes training a computational model, using the training images and the labels, to associate runtime images with a stage of the multiple stages based on characteristics of one or more surgical tools that are depicted by the runtime images. Another method includes associating, using a computational model, runtime images with a stage of a surgical procedure based on characteristics of one or more surgical tools depicted by the runtime images and generating output that indicates the stage associated with each of the runtime images.

### CROSS REFERENCE TO RELATED APPLICATION

The present application is a non-provisional application claiming priority to U.S. provisional application No. 63/375,174, filed on Sep. 9, 2022, the contents of which are hereby incorporated by reference.

### BACKGROUND

Video recordings of surgical procedures can be used for educational or documentation purposes. For example, a surgeon in training can view such videos to learn proper surgical techniques. Often these videos are too long and include too much non-informative footage such that they have little educational or clinical value. These video files can also be large and take up too much space on digital storage media.

### SUMMARY

A first example includes a method comprising: accessing training images that collectively depict multiple stages of a surgical procedure; accessing labels that indicate, for each of the training images, characteristics of one or more surgical tools depicted and a stage of the multiple stages of the surgical procedure depicted; and training a computational model, using the training images and the labels, to associate runtime images with a stage of the multiple stages based on characteristics of one or more surgical tools that are depicted by the runtime images.

A second example includes a non-transitory computer readable medium storing instructions that, when executed by one or more processors of a computing device, cause the computing device to perform the method of the first example.

A third example includes a computing device comprising: one or more processors and a computer readable medium storing instructions that, when executed by the one or more processors, cause the computing device to perform the method of the first example.

A fourth example includes a method comprising: associating, using a computational model, runtime images with a stage of a surgical procedure based on characteristics of one or more surgical tools depicted by the runtime images; and generating output that indicates the stage associated with each of the runtime images.

A fifth example includes a non-transitory computer readable medium storing instructions that, when executed by one or more processors of a computing device, cause the computing device to perform the method of the fourth example.

A sixth example includes a computing device comprising: one or more processors and a computer readable medium storing instructions that, when executed by the one or more processors, cause the computing device to perform the method of the fourth example.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/−0-5% of the recited value.

The following publications are hereby incorporated by reference: Daniel King, “Automatic Summarization of Endoscopic Surgical Videos,” http://hdl.handle.net/1773/48933; King D, Adidharma L, Peng H, Moe K, Li Y, Yang Z, Young C, Ferreria M, Humphreys I, Abuzeid W M, Hannaford B, Bly R A. Automatic summarization of endoscopic skull base surgical videos through object detection and hidden Markov modeling. Comput Med Imaging Graph. 2023 September; 108:102248. doi: 10.1016/j.compmedimag.2023.102248. Epub 2023 May 25. PMID: 37315397.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a block diagram of a computing device, according to an example.

FIG. 2 is a schematic diagram of training images, according to an example.

FIG. 3 is a schematic diagram of a hidden Markov model (HMM), according to an example.

FIG. 4 is a schematic diagram of runtime images, according to an example.

FIG. 5 is a block diagram of a method, according to an example.

FIG. 6 is a block diagram of a method, according to an example.

### DETAILED DESCRIPTION

This disclosure includes systems and methods for processing images to associate the images with stages of a surgical procedure, such as an approach stage, an operation stage, and a reconstruction stage. This can be one of several steps for generating a condensed version of a video depicting a surgical procedure. The condensed video is shorter than the initially recorded video and includes a higher percentage of video frames that are informative. For example, the condensed video may be generated by discarding video frames that depict mundane tasks such as tool cleaning, blurry video frames, or video frames that depict areas that are external to the patient. The process of generating the condensed video is also aided by processing the images to associate the images with stages of a surgical procedure, as described below.

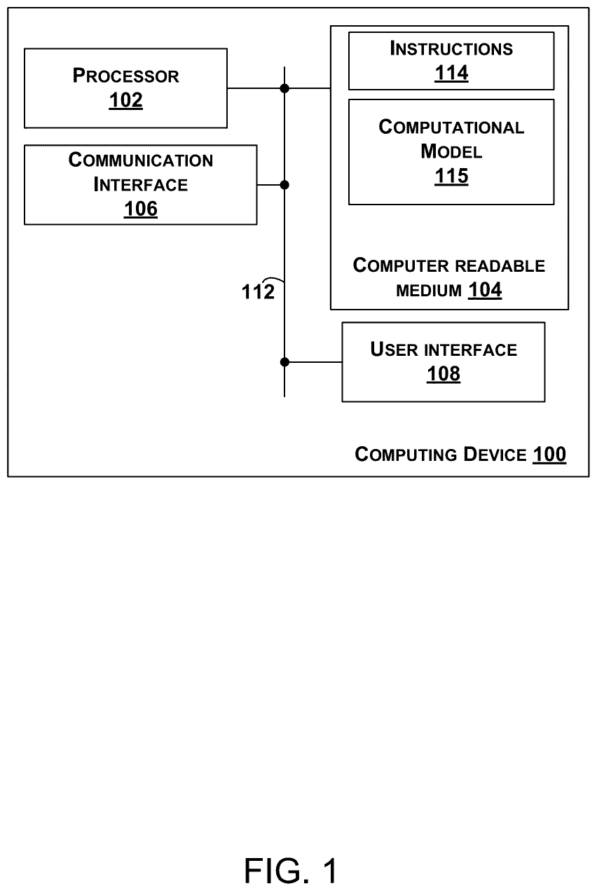

FIG. 1 is a block diagram of a computing device 100. The computing device 100 includes one or more processors 102, a non-transitory computer readable medium 104, a communication interface 106, and a user interface 108. Components of the computing device 100 are linked together by a system bus, network, or other connection mechanism 112.

The one or more processors 102 can be any type of processor(s), such as a microprocessor, a field programmable gate array, a digital signal processor, a multicore processor, etc., coupled to the non-transitory computer readable medium 104.

The non-transitory computer readable medium 104 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

The non-transitory computer readable medium 104 stores instructions 114. The instructions 114 are executable by the one or more processors 102 to cause the computing device 100 to perform any of the functions or methods described herein. The non-transitory computer readable medium 104 also stores a computational model 115, which can take the form of a convolutional neural network (CNN) and/or a hidden Markov model (HMM), for example.

The communication interface 106 can include hardware to enable communication within the computing device 100 and/or between the computing device 100 and one or more other devices. The hardware can include any type of input and/or output interfaces, a universal serial bus (USB), PCI Express, transmitters, receivers, and antennas, for example. The communication interface 106 can be configured to facilitate communication with one or more other devices, in accordance with one or more wired or wireless communication protocols. For example, the communication interface 106 can be configured to facilitate wireless data communication for the computing device 100 according to one or more wireless communication standards, such as one or more Institute of Electrical and Electronics Engineers (IEEE) 801.11 standards, ZigBee standards, Bluetooth standards, etc. As another example, the communication interface 106 can be configured to facilitate wired data communication with one or more other devices. The communication interface 106 can also include analog-to-digital converters (ADCs) or digital-to-analog converters (DACs) that the computing device 100 can use to control various components of the computing device 100 or external devices.

The user interface 108 can include any type of display component configured to display data. As one example, the user interface 108 can include a touchscreen display. As another example, the user interface 108 can include a flat-panel display, such as a liquid-crystal display (LCD) or a light-emitting diode (LED) display. The user interface 108 can include one or more pieces of hardware used to provide data and control signals to the computing device 100. For instance, the user interface 108 can include a mouse or a pointing device, a keyboard or a keypad, a microphone, a touchpad, or a touchscreen, among other possible types of user input devices. Generally, the user interface 108 can enable an operator to interact with a graphical user interface (GUI) provided by the computing device 100 (e.g., displayed by the user interface 108).

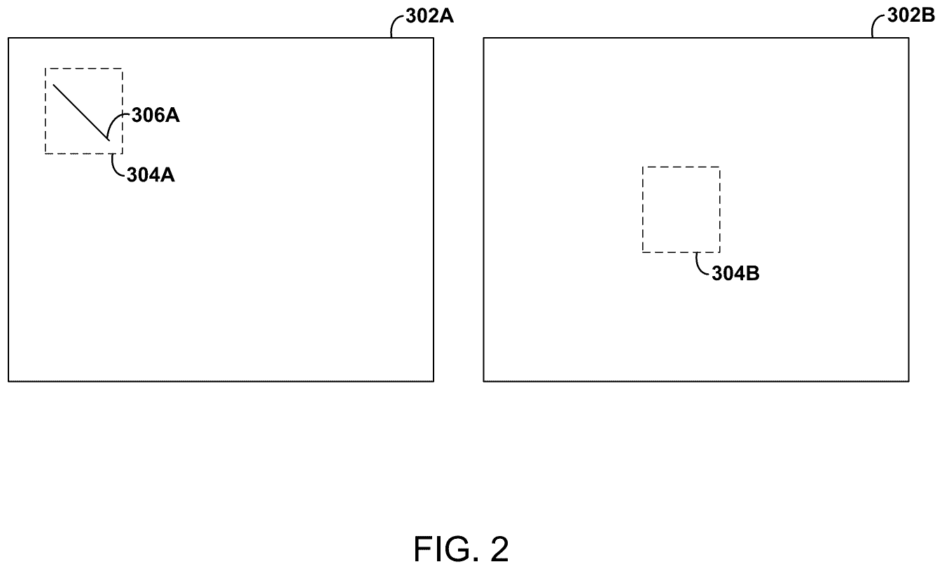

FIG. 2 is a schematic diagram of a training image 302A and a training image 302B. The training image 302A and the training image 302B are representative of many training images (e.g., hundreds or thousands of training images) that collectively depict or are otherwise captured during multiple stages of one or more instances of the same type of surgical procedure. For example, the training images can collectively depict or be captured during the approach stage, the operation stage, or the reconstruction stage of one or more endoscopic nasal surgeries.

Training images can be obtained from one or more imaging devices that operate during surgery. Example imaging devices include monocular or stereo surgical endoscopes, wireless cameras deployed inside the patient, cameras imaging the proximal portion of instruments lying outside the patient, or cameras imaging the surgical field from overhead lights, surgeon body cameras, or surgeon loupes. If surgery is conducted with the aid of intraoperative CT or MM scanning, the training images could come from one or more of those devices.

Each of the training images are associated with one or more labels that indicate characteristics of the training image. In some examples, a user such as a surgeon generates the labels that characterize the training images. For instance, the user generates input via the user interface 108. Such input characterizes the training images by indicating one or more characteristics such as: whether any surgical tool appears within the training images; a type, a position, and/or an orientation of the surgical tool(s) if present in the training images; and whether the training images depict an area of interest within the patient such as a nasal cavity or instead depict an area outside of the patient such as an operating room floor. The input is stored as labels in the form of metadata that is associated with each training image.

Accordingly, the computing device 100 accesses many training images that collectively depict or were otherwise captured during multiple stages of a surgical procedure, including the training image 302A and the training image 302B. For instance, the training image 302A and the training image 302B depict or were otherwise captured during the approach stage, the operation stage, and the reconstruction stage of one or more endoscopic nasal surgeries.

The approach stage generally includes actions related to navigating an endoscope and one or more surgical tools to a site within the patient that is in need of surgery. For example, this can involve navigating through the nasal cavity and removing tissue and bone to enhance tool access to the surgical field. The operation stage typically involves the primary surgical task such as resection of diseased tissue or mass. During the reconstruction stage, the primary task is to approximate remaining anatomy in the surgical field to a functional baseline. For example, after removal of an intracranial mass (operation stage), the reconstruction of the barrier between the intracranial and sinus spaces is re-established using synthetic or patient derived autologous tissue such as bone or mucosa and fixed in position.

The computing device 100 also accesses many labels including the label 304A and the label 304B. The labels indicate, for each of the training images, one or more characteristics such as whether any surgical tool appears within the training images; a type, a position, and/or an orientation of the surgical tool(s) if present in the training images; and whether the training images depict an area of interest within the patient such as a nasal cavity or instead depict an area outside of the patient such as an operating room floor.

For example, the computing device 100 accesses the label 304A that indicates that the training image 302A depicts a surgical tool 306A in the form of a forceps. The label 304A also indicates the pose of the surgical tool 306A as (1) a position of the surgical tool 306A within the training image 302A with two-dimensional coordinates and (2) an orientation of the surgical tool 306A within the training image 302A with an angle. The label 304A can further indicate that the training image 302A depicts the operation stage of the surgical procedure. In other examples, the label 304A or an additional label indicates the pose of one or more of a suction tool, a drill, a ring curette, a rongeur, a scissors, or a cauterizer that is depicted by the training image 302A. In other variations, the label 304A can indicate that the training image 302A depicts the approach stage or the reconstruction stage of the surgical procedure. In addition, the label 304A can also indicate whether the training image 302A depicts an area of interest within the patient or instead depicts an area outside of the patient.

Additionally or alternatively, the computing device 100 accesses the label 304B that indicates that the training image 302B does not depict a surgical tool. The label 304B can further indicate that the training image 302B depicts or was captured during the approach stage of the surgical procedure. In other examples, the label 304B can indicate that the training image 302B depicts or was captured during the operation stage or the reconstruction stage of the surgical procedure. In addition, the label 304B can also indicate whether the training image 302B depicts an area of interest within the patient or instead depicts an area outside of the patient.

The computing device 100 then trains the computational model 115, using the training images and the labels, to associate runtime images with a particular stage of the surgical procedure, based on one or more characteristics such as: a type and a pose of one or more surgical tools that are depicted by the runtime images, whether the runtime images include a surgical tool, or whether the runtime images depict an area within the patient. The runtime images represent unlabeled images of a surgical procedure that are captured after the computational model 115 has been trained and/or finalized. That is, the finalized computational model 115 is used to classify unlabeled images of a new surgical procedure into stages of the surgical procedure.

For example, the computing device 100 adjusts one or more hidden layers of the computational model 115 (e.g., a convolutional neural network (CNN) and/or a hidden Markov model (HMM)) such that when the training image 302A is provided as an input to the computational model 115, the computational model 115 is more likely to generate an output indicating that the training image 302A is associated with the stage of the surgical procedure indicated by the label 304A or other labels associated with the training image 302A. In one aspect, the computational model 115 is adjusted to better represent a correlation between (1) characteristics of the training image 302A indicated by the label 304A or other labels associated with the training image 302A and (2) the stage of the surgical procedure indicated by the label 304A or other labels associated with the training image 302A. In this example, the characteristics of the training image 302A can include one or more of whether a surgical tool is present; the type, the position, and/or the orientation of one or more depicted surgical tools, and whether the training image 302A depicts an area within the patient such as a nasal cavity or instead depicts an area outside of the patient such as an operating room floor.

Similarly, the computing device 100 adjusts one or more hidden layers of the computational model 115 such that when the training image 302B is provided as an input to the computational model 115, the computational model 115 is more likely to generate an output indicating that the training image 302B is associated with the stage of the surgical procedure indicated by the label 304B or other labels associated with the training image 302B. In one aspect, the computational model 115 is adjusted to better represent a correlation between (1) characteristics of the training image 302B indicated by the label 304B or other labels associated with the training image 302B and (2) the stage of the surgical procedure indicated by the label 304B or other labels associated with the training image 302B. In this example, the characteristics of the training image 302B can include one or more of whether a surgical tool is present; the type, the position, and/or the orientation of one or more depicted surgical tools, and whether the training image 302B depicts an area within the patient such as a nasal cavity or instead depicts an area outside of the patient such as an operating room floor.

More particularly, the computing device 100 iteratively adjusts the hidden layers of the computational model 115 such that the computational model 115 can recognize characteristics of unlabeled runtime images, such as one or more of whether a surgical tool is present in the runtime images; the type, the position, and/or the orientation of one or more depicted surgical tools, and whether the runtime image depicts an area within the patient such as a nasal cavity or instead depicts an area outside of the patient such as an operating room floor. The computational model 115 also associates these combinations of characteristics of the runtime images such that the runtime images can be assigned to a stage of the surgical procedure.

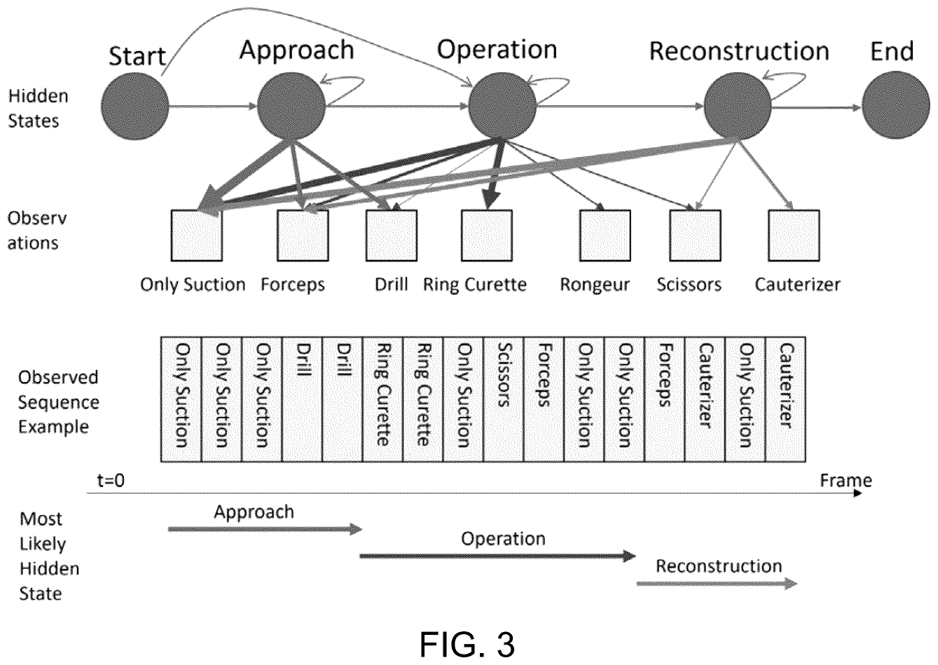

In some examples, training the computational model 115 includes training a hidden Markov model (HMM) to associate the runtime images with a stage of the surgical procedure based on the type and the pose of the one or more surgical tools depicted by the runtime images. In the HMI, the “hidden states” are the stages of the surgical procedure, such as the approach stage, the operation stage, and the reconstruction stage. The possible observations of the HMM with respect to the runtime images are: only the suction tool is present, the forceps is present, the drill is present, the ring curette is present, an unknown tool is present, a rongeur is present, a scissors is present, and a cauterizer is present. The structure of the HMM is shown in FIG. 3.

In some examples, the HMM is trained using an unsupervised Baum-Welch algorithm. More particularly, training the HMM includes iteratively refining estimated probabilities of observing a particular type of surgical tool during a particular stage of the surgical procedure. Training the HMI can also include iteratively refining estimated probabilities of transiting from one stage of the surgical procedure to another stage of the surgical procedure.

The computational model 115 can also be trained to associate the runtime images with the stages such that the runtime images associated with the approach stage are captured prior to the runtime images associated with the operation stage, which are captured prior to the runtime images associated with the reconstruction stage. For example, the computational model 115 can use timestamps that correspond with the runtime images to put the runtime images in chronological order and then assign the runtime images to stages of the surgical procedure at least on part based on the assumption that the approach stage precedes the operation stage, which precedes the reconstruction stage.

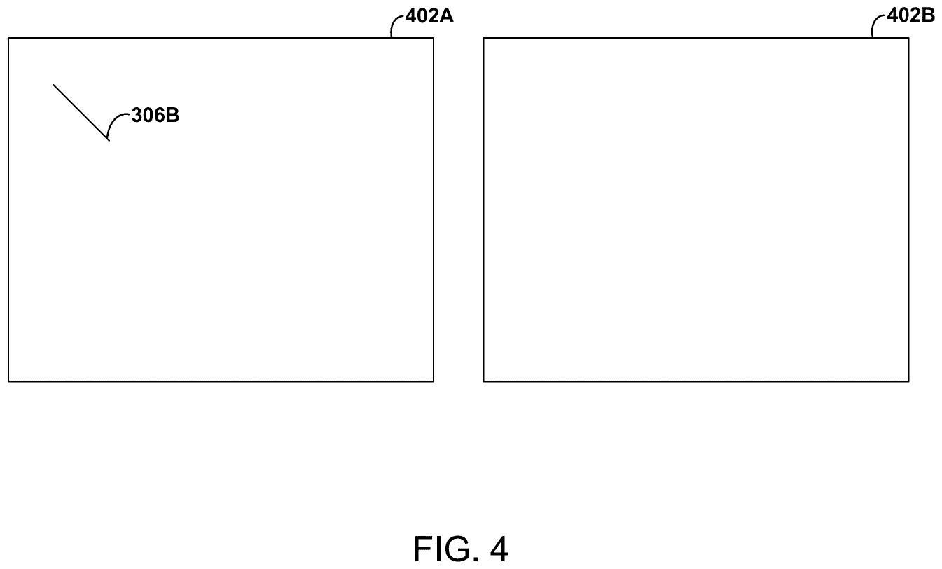

FIG. 4 is a schematic diagram of a runtime image 402A and a runtime image 402B. The computing device 100 uses the computational model 115 to associate many runtime images, including the runtime image 402A and the runtime image 402B, with a stage of a surgical procedure depicted by the runtime images, based on one or more characteristics such as: a type and/or a pose of one or more surgical tools that are depicted by the runtime images, whether the runtime images include a surgical tool, or whether the runtime images depict an area within the patient. The user interface 108 then generates output that indicates the stage associated with each of the runtime images.

In some examples, prior to associating the runtime images with stages of the surgical procedure, the computing device 100 identifies the runtime images from a set of images such that the runtime images depict a surgical tool and the other images do not depict a surgical tool. For example, the computing device 100 identifies the runtime image 402A as depicting a surgical tool 306B and identifies the runtime image 402B as not depicting a surgical tool. In this example, the computational model 115 is trained using only images depicting a surgical tool and is provided input only in the form of images that show a surgical tool. That is, the computational model 115 is provided the runtime image 402A as input but not the runtime image 402B.

In various examples, the computing device 100 uses the CNN to determine one or more characteristics such as the type of the one or more surgical tools depicted by the runtime images and associates the runtime images with respective stages of the surgical procedure accordingly. Furthermore, the computing device 100 uses the CNN to determine the pose of the one or more surgical tools depicted by the runtime images and associates the runtime images with respective stages of the surgical procedure accordingly.

In some examples, the computing device 100 also uses the HMM to associate the runtime images with respective stages of the surgical procedure based on the type of the one or more surgical tools depicted by the runtime images. Furthermore, the computing device 100 uses the HMM to associate the runtime images with respective stages of the surgical procedure based on the pose of the one or more surgical tools depicted by the runtime images.

In some embodiments, some or all parameters of the HMM can be initialized based on human expert knowledge. For example, if typical durations are known (by human experts) for the surgical stages, values can be computed for the parameters of the HMM state-transition matrix which initialize the model to be likely to produce those durations. Similar encodings of expert information can be performed for the expected type and location of each surgical instrument in each surgical stage. It is known to those in the art that pre-initializing HMM parameters as described here can improve the performance of the Baum-Welch algorithm compared to initializing HMM parameters with random numbers.

In common examples, the computing device 100 associates a runtime image with an operation stage of the surgical procedure based on detecting a ring curette or a rongeur within the runtime image.

In common examples, the computing device 100 associates a runtime image with a reconstruction stage of the surgical procedure based on detecting a cauterizer within the runtime image.

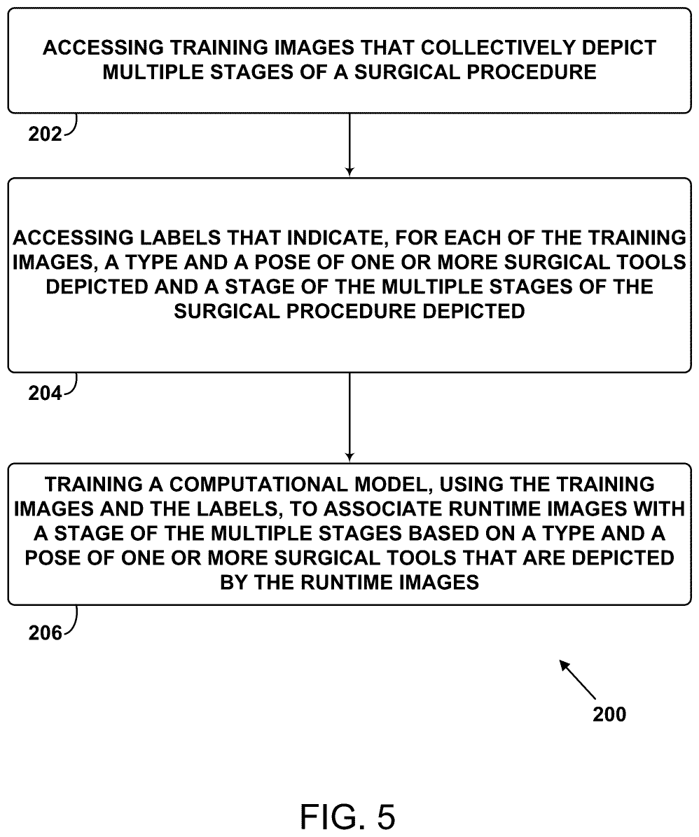

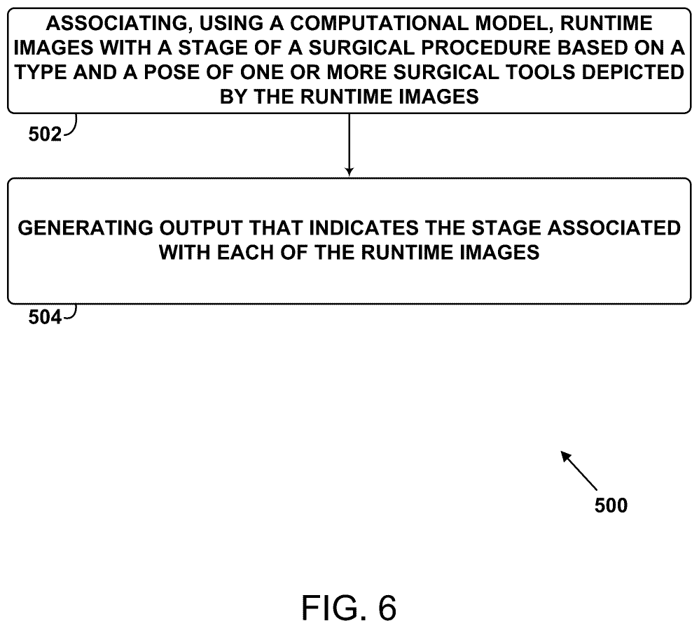

FIG. 5 and FIG. 6 are block diagrams of a method 200 for training the computational model 115 and a method 500 for using the computational model to associate runtime images with stages of a surgical procedure. As shown in FIG. 5 and FIG. 6, the method 200 and the method 500 include one or more operations, functions, or actions as illustrated by blocks 202, 204, 206, 502, and 504. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 202, the method 200 includes the computing device 100 accessing the training images 302 that collectively depict multiple stages of a surgical procedure. Functionality related to block 202 is discussed above with reference to FIG. 2 and FIG. 3.

At block 204, the method 200 includes the computing device 100 accessing the labels 304 that indicate, for each of the training images 302, characteristics of one or more surgical tools 306 depicted and a stage of the multiple stages of the surgical procedure depicted. Functionality related to block 204 is discussed above with reference to FIG. 2 and FIG. 3.

At block 206, the method 200 includes the computing device 100 training the computational model 115, using the training images 302 and the labels 304, to associate the runtime images 402 with a stage of the multiple stages based on characteristics of one or more surgical tools 306 that are depicted by the runtime images 402. Functionality related to block 206 is discussed above with reference to FIGS. 2-4.

At block 502, the method 500 includes the computing device 100 associating, using the computational model 115, the runtime images 402 with a stage of a surgical procedure based on characteristics of one or more surgical tools 306 depicted by the runtime images 402. Functionality related to block 502 is discussed above with reference to FIG. 4.

At block 504, the method 500 includes the computing device 100 generating output hat indicates the stage associated with each of the runtime images 402. Functionality related to block 504 is discussed above with reference to FIG. 4.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

What is claimed is:

1. A method comprising:
accessing training images that collectively depict multiple stages of a surgical procedure;
accessing labels that indicate, for each of the training images, characteristics of one or more surgical tools depicted and a stage of the multiple stages of the surgical procedure depicted; and
training a computational model, using the training images and the labels, to associate runtime images with a stage of the multiple stages based on characteristics of one or more surgical tools that are depicted by the runtime images.

2. The method of claim 1, wherein the training images are first training images and the labels are first labels, the method further comprising, prior to training the computational model:
accessing the first training images and second training images that do not depict a surgical tool;
accessing second labels that indicate, for each of the second training images, that a surgical tool is not depicted; and
training the computational model, using the first training images, the second training images, the first labels, and the second labels, to classify the runtime images according to whether the runtime images include a surgical tool.

3. The method of claim 1, wherein the training images are first training images and the labels are first labels that indicate that each of the first training images depicts an area within a patient, the method further comprising, prior to training the computational model:
accessing the first training images and second training images;
accessing second labels that indicate, for each of the second training images, that an area within the patient is not depicted; and
training the computational model, using the first training images, the second training images, the first labels, and the second labels, to classify the runtime images according to whether the runtime images depict an area within the patient.

4. The method of claim 1, wherein accessing the labels comprises accessing a label that indicates a pose of one or more of a suction tool, a forceps, a drill, a ring curette, a rongeur, a scissors, or a cauterizer.

5. The method of claim 1, wherein accessing the labels comprises accessing a label that corresponds to an approach stage, an operation stage, or a reconstruction stage of the surgical procedure.

6. The method of claim 1, wherein training the computational model comprises training a convolutional neural network (CNN) to determine a type and a pose of the one or more surgical tools present within the runtime images.

7. The method of claim 1, wherein training the computational model comprises training a hidden Markov model (HMM) to associate the runtime images with a stage of the multiple stages based on characteristics of the one or more surgical tools depicted by the runtime images.

8. The method of claim 7, wherein training the HMM comprises training the HMM with an unsupervised Baum-Welch algorithm.

9. The method of claim 7, wherein training the HMM comprises training the HMM by iteratively refining an estimated probability of observing a particular type of surgical tool during a particular stage of the surgical procedure.

10. The method of claim 7, wherein training the HMM comprises training the HMM by iteratively refining an estimated probability of transiting to a first stage of the surgical procedure from a second stage of the surgical procedure.

11. The method of claim 1, wherein the multiple stages include an approach stage, an operation stage, and a reconstruction stage, and wherein training the computational model comprises training the computational model to associate the runtime images such that the runtime images associated with the approach stage are captured prior to the runtime images associated with the operation stage which are captured prior to the runtime images associated with the reconstruction stage.

12. A method comprising:
associating, using a computational model, runtime images with a stage of a surgical procedure based on characteristics of one or more surgical tools depicted by the runtime images; and
generating output that indicates the stage associated with each of the runtime images.

13. The method of claim 12, further comprising, prior to the associating, identifying the runtime images from among a set of images such that the runtime images depict a surgical tool, wherein associating the runtime images comprises associating the runtime images identified as depicting a surgical tool.

14. The method of claim 12, further comprising using a convolutional neural network (CNN) to determine a type of the one or more surgical tools depicted by the runtime images, wherein associating the runtime images comprises associating the runtime images based on the type of the one or more surgical tools depicted by the runtime images.

15. The method of claim 12, further comprising using a convolutional neural network (CNN) to determine a pose of the one or more surgical tools depicted by the runtime images, wherein associating the runtime images comprises associating the runtime images based on the pose of the one or more surgical tools depicted by the runtime images.

16. The method of claim 12, wherein associating the runtime images comprises using a hidden Markov model (HMM) to associate the runtime images based on a type of the one or more surgical tools depicted by the runtime images.

17. The method of claim 12, wherein associating the runtime images comprises using a hidden Markov model (HMM) to associate the runtime images based on a pose of the one or more surgical tools depicted by the runtime images.

18. The method of claim 12, wherein associating the runtime images comprises associating a runtime image with an operation stage of the surgical procedure based on detecting a ring curette or a rongeur within the runtime image.

19. The method of claim 12, wherein associating the runtime images comprises associating a runtime image with a reconstruction stage of the surgical procedure based on detecting a cauterizer within the runtime image.

20. A non-transitory computer readable medium storing instructions that, when executed by one or more processors of a computing device, cause the computing device to perform functions comprising:
associating, using a computational model, runtime images with a stage of a surgical procedure based on characteristics of one or more surgical tools depicted by the runtime images; and
generating output that indicates the stage associated with each of the runtime images.
